(19) United States
(12) Patent Application Publication
(10) Pub. No.: US 2014/0107719 A1
(43) Pub. Date: Apr. 17, 2014

(54) SYSTEMS AND METHODS FOR POSTEXTRASYSTOLIC POTENTIATION USING ANODIC AND CATHODIC PULSES GENERATED BY AN IMPLANTABLE MEDICAL DEVICE

(71) Applicant: PACESETTER, INC., Sylmar, CA (US)

(72) Inventors: Gene A. Bornzin, Simi Valley, CA (US); Kyungmoo Ryu, Palmdale, CA (US)

(73) Assignee: PACESETTER, INC., Sylmar, CA (US)

(21) Appl. No.: 13/649,657

(22) Filed: Oct. 11, 2012

(51) Int. Cl. A61N 1/362

(52) U.S. Cl. 607/9

(57) ABSTRACT

Techniques are provided for use with implantable medical devices to deliver paired or coupled postextrasystolic potentiation (PESP) pacing using split or bifurcated anodic and cathodic pulses. In a paired pacing example, a single-phase anodic pulse is delivered by the device that has sufficient amplitude to depolarize and contract myocardial tissue. During or just following a subsequent relative refractory period, a single-phase cathodic stimulation pulse is delivered that has sufficient amplitude to depolarize but not contract myocardial tissue, i.e., the cathodic pulse provides for PESP. In a coupled pacing example, the single-phase anodic pulse is delivered during or just following the relative refractory period of a first cardiac cycle; whereas the single-phase cathodic pulse is delivered during or immediately following the relative refractory period of the next consecutive cardiac cycle.

### CROSS REFERENCE TO RELATED APPLICATIONS

This application is related to U.S. patent application Ser. No. ______, filed concurrently herewith, titled “Systems and Methods for Packed Pacing Using Bifurcated Pacing Pulses of Opposting Polarity Generated by an Implantable Medical Device” (Atty Docket A12P1046).

### FIELD OF THE INVENTION

The invention generally relates to implantable cardiac stimulation devices such as pacemakers and implantable cardioverter-defibrillators (ICDs) and, in particular, to techniques for delivering postextrasystolic potentiation (PESP) therapy.

### BACKGROUND OF THE INVENTION

PESP therapy is a pacing therapy wherein extra stimulation pulses are delivered by a pacemaker or other suitable device during or immediately beyond a relative refractory period following paced or intrinsic depolarization. The extra PESP stimulus causes the heart muscle to depolarize a second time but does not cause significant contraction of the muscle. The second depolarization acts on the sarcoplasmic reticulum to release an additional bolus of calcium. It is generally believed that the additional intracellular calcium ions provide for increased contractility. Another consequence of the extra stimulus provided during or just following the relative refractory period is to extend the overall refractory interval, which slows the heart and allows the pacemaker to control the heart rate. During actual delivery of PESP pulses, as with all stimulation pulses, the pacemaker blanks or blocks its sensing channels so as not to misinterpret the electrical stimulus as being an intrinsic electrical signal (i.e. an electrical signal arising from the myocardial tissue.)

Note that, following a paced or intrinsic depolarization, pacemakers typically track a refractory interval that includes both an absolute refractory period and a subsequent relative refractory period. During the absolute refractory period, a second myocardial depolarization cannot be triggered, regardless of the amplitude of extra stimulus, because the myocardial tissue is not susceptible to further electrical stimulus at that time. Hence, PESP pulses are not delivered during the absolute refractory period. During or just following the subsequent relative refractory period, a second depolarization can be triggered with a sufficiently large stimulation pulse. However, it should be noted that there is no sharp delineation between the relative refractory period and the non-refractory period. The threshold asymptotically approaches a minimum at what is known as the late diastolic threshold. The thresholds increase slightly as the cycle length shortens and then, at the relative refractory period, the thresholds start to climb dramatically into an absolute refractory period. Accordingly, PESP pulses are typically delivered late in or just “outside” the relative refractory period. So long as the pulses are timed to generate closely spaced dual-depolarization, it is deemed as successful PESP. Pulse amplitude may be adjusted to achieve capture at the outer edge of the relative refractory period. The pulse amplitude need only be slightly larger than the diastolic threshold to achieve capture. Accordingly, PESP pulses are typically delivered during or just following the relative refractory period using a stimulation pulse of nominal pulse amplitude to trigger depolarization without contraction.

There are various applications for PESP therapy. PESP may be used to enhance cardiac resynchronization therapy (CRT) by increasing contractility beyond what is typically achieved by merely restoring synchrony. PESP may be used to slow the ventricles during atrial fibrillation (AF) because PESP prolongs the refractory interval. That is, the additional depolarization during the relative refractory period caused by the PESP pulse has the effect of extending the overall refractory interval. The longer refractory interval acts to block the conduction of rapid atrial impulses associated with AF. PESP thus can provide for rate control during AF. A secondary benefit may be enhanced contractility for patients with AF and heart failure. Further, PESP may be used to treat patients with low ejection fraction (EF) and narrow QRS heart failure (i.e. a form of heart failure wherein the electrical signals associated with ventricular depolarization (QRS complexes) are shorter than usual.) PESP may be used to treat their cardiac insufficiency. Still further, PESP may be used to treat heart failure with preserved EF. Patients with heart failure with preserved EF can benefit because PESP enhances the rate of relaxation.

PESP can be implemented in accordance with either “paired pacing” or “coupled pacing” techniques. With paired pacing, the additional PESP pulse is delivered during or just beyond the relative refractory period following a paced depolarization. With coupled pacing, the additional stimulation is delivered the relative refractory period following an intrinsic depolarization. Paired and coupled pacing is discussed in U.S. Published Patent Application No. 2010/0094371 of Bornzin et al., entitled “Systems and Methods for Paired/Coupled Pacing” and in U.S. patent application Ser. No. 11/929,719, also of Bornzin et al., filed Oct. 30, 2007, entitled “Systems and Methods for Paired/Coupled Pacing and Dynamic Overdrive/Underdrive Pacing.”

Typically, when PESP is implemented using paired pacing, two otherwise conventional stimulation pulses are delivered—a primary pulse intended to trigger myocardial contraction and a secondary (PESP) pulse intended to improve contractility. Each pulse is a bipolar pulse that consists of a cathodic pulse/phase (of typically 0.1 to 2 milliseconds (ms) in duration) followed by a second pulse/phase, known as the “rapid recharge” or “discharge” phase, which includes an anodic pulse typically 4 to 25 ms in duration. The rapid recharge phase restores the charge that was delivered during the cathodic output phase. A consequence of the relatively long anodic phase is that device sensing on corresponding sensing channels is blocked or blanked for a relatively long period of time, which can interfere with the detection of events such as premature ventricular contractions (PVCs.)

FIG. 1 illustrates a conventional circuit 2 for generating stimulation pulses, including stimulation pulses and PESP pulses. The operation of the circuit will be summarized with respect to the delivery of the initial (primary) pacing pulse but it should be understood that the same procedure is conventionally employed for the delivery of the subsequent (secondary) PESP pulse during the relative refractory period. Charge for delivering the stimulation pulse is held in a pacing charge capacitor. A separate charge coupling capacitor blocks direct current to the tip/ring electrodes during pacing and thus avoids electrode corrosion. Assuming the pacing charge capacitor has been properly charged from the voltage source V (e.g. a battery), the delivery of the stimulation pulse consists of two steps: “pacing” and “recharge.” During pacing, a first transistor switch, SWpace, is configured to deliver the cathodic phase of the stimulation pulse, which is of a sufficient voltage amplitude and duration to affect stimulation of the heart (i.e. depolarization and contraction.) More specifically, SWpace is closed to provide a path for charge to flow from the pacing capacitor into the coupling capacitor through the pacing tip and ring electrodes via heart tissue (which is represented by resistance R.) During this cathodic process, the coupling capacitor (typically 5 microfarads) accumulates a small amount of charge, Q=CΔV, subject to a small voltage, ΔV, which is only a fraction of the voltage of supply V. The cathodic phase terminates by opening the delivering transistor switch, SWpace.

The charge that accumulated on the coupling capacitor during the cathodic phase is then taken off the coupling capacitor during the anodic phase by promptly closing the recharge switch (SWrecharge) for 10 to 25 ms. This anodic phase is also called recharge (or discharge). 10 to 25 ms is usually more than sufficient time to discharge the capacitor through the pacing load, R, which is typically in the range of 500 ohms. The time constant for the recharge is 500 ohms*5 microfarads 2.5 ms. Therefore, 10 to 25 ms is 4 to 10 time constants. Note that a passive recharge resistor is often provided across the SWrecharge switch. The passive recharge resistor has a relatively high resistance of about 40 kilo-ohms to allow for dissipation of any residual charge during the subsequent absolute refractory period. Also, during the absolute refractory period, the charging switch is controlled to recharge the pacing charge capacitor from the voltage source for delivery of the PESP stimulation pulse. Thereafter, upon completion of the absolute refractory period triggered by the initial stimulation pulse, the overall process is repeated during the relative refractory period to deliver the PESP pulse, which likewise includes both cathodic and anodic phases. Note that the various switches of the circuit are controlled by a microcontroller or other suitable control system (not shown in FIG. 1) of the pacing device. Note also that this is a simplified pacing circuit that only illustrates circuit components pertinent to this discussion. State-of-the-art pacing circuits can include numerous additional components.

FIG. 2 illustrates the voltage shape of a typical biphasic primary stimulation pulse or biphasic secondary PESP pulse delivered via the circuit of FIG. 1, including a cathodic pulse/phase 3 and a longer anodic pulse/phase 4. During the initial cathodic phase, SWpace is closed while SWrecharge is open. During the anodic recharge (or discharge) phase, SWrecharge is closed while SWpace open. As noted, typical cathodic stimulation pulse/phases are within the range of 0.1 to 2 ms while the anodic recharge pulse/phases are within the range of 4 to 25 ms, yielding a total pulse duration of typically at least 6 ms up to about to 27 ms. During this period of time, denoted by reference numeral 5, the corresponding sensing channels are blanked or blocked, preventing detection of cardioelectric events such as PVCs. Conventionally, both the initial stimulation pulse and the subsequent PESP pulse have this two phase (i.e. biphasic) shape.

FIG. 3 illustrates a pair of biphasic stimulation pulses—a pacing pulse 6 and a PESP pulse 8—separated by a refractory interval that includes an absolute refractory period and a relative refractory period. The PESP pulse is delivered during the relative refractory period. Sense channel blanking intervals are also shown, which correspond (at least) to the duration of the stimulation pulses.

It would be desirable to provide improved techniques for delivering PESP therapy that reduce the amount of time in which sensing is blanked or that provide other advantages, and it is to this end that various aspects of the invention are directed.

### SUMMARY OF THE INVENTION

In an exemplary embodiment, a method is provided for use with an implantable cardiac stimulation device equipped to deliver PESP pacing. In accordance with an exemplary paired pacing method, a single-phase primary stimulation pulse is generated for delivery to the heart of the patient. The single-phase primary pulse (e.g., anodic) has sufficient pulse amplitude and width to depolarize and contract myocardial tissue. A refractory interval is tracked within the heart of the patient subsequent the single-phase primary stimulation pulse that includes, at least, a relative refractory period. A single-phase secondary stimulation pulse is then generated for delivery to the heart of the patient during or immediately following the relative refractory period (e.g. within 50 milliseconds from the end of the relative refractory period.) The secondary stimulation pulse is opposite in polarity to the primary pulse (e.g., cathodic) and is configured to achieve PESP (i.e. the pulse amplitude and width of the secondary stimulation pulse are set to depolarize but not contract the myocardial tissue.)

Hence, rather than delivering a pair of biphasic pulses—each having cathodic and anodic pulse phases—as with predecessor paired pacing techniques, the exemplary method splits or bifurcates a single stimulation pulse into two pulses/phases separated by the absolute refractory period, the first anodic pulse being sufficient to trigger contraction of myocardial tissue, the second cathodic pulse being sufficient to induce PESP. By splitting a single biphasic pulse into separate anodic/cathodic single-phase pulses, the amount of time during which sensing is blanked or blocked can be reduced. In one particular embodiment, the single-phase anodic and cathodic pulses each have absolute pulse amplitudes of 2.0 V and widths of about 1 ms in duration, significantly reducing the amount of time needed to blank the corresponding sensing channel as compared to the predecessor techniques discussed above. The total charge consumed during this exemplary split phase stimulation process (also referred to herein as “dual phase” process) is equivalent to a single 4.0 V pulse with a 1.0 ms duration. This is quite efficient in terms of energy but since anodic thresholds are slightly higher than cathodic pulses, the charge consumed by this process may be slightly higher than conventional pacing processes.

In accordance with an exemplary coupled pacing method, a first single-phase stimulation pulse is generated for delivery to the heart during or immediately following the relative refractory period following an intrinsic depolarization (i.e. an R-wave or QRS complex.) The first single-phase pulse may be anodic and has sufficient pulse width and amplitude to depolarize myocardial tissue and induce PESP. During the next cardiac cycle, after detection of another intrinsic depolarization, a second single-phase stimulation pulse is generated for delivery during or immediately following the corresponding relative refractory period. The second single-phase stimulation pulse is opposite in polarity to the first (e.g. cathodic) but likewise has sufficient pulse width and amplitude to depolarize myocardial tissue and induce PESP. Hence, the polarity of the single-phase PESP pulses alternates from one cardiac cycle to the next. In this manner, a single biphasic pulse is split or bifurcated during coupled pacing into two pulses/phases for delivery during consecutive cardiac cycles, the first pulse being anodic and the second pulse being cathodic.

In an exemplary embodiment where the implanted device is a pacemaker, ICD or CRT device, the device is equipped for both paired pacing and coupled pacing. The device preferably employs a pacing circuit to deliver pacing pulses and PESP pulses that has at least one capacitor (e.g. a coupling capacitor) and at least one passive recharge resistor. For paired pacing, during the absolute refractory period after the primary pulse, the passive recharge resistor is switched out of the circuit so that the capacitor does not lose its charge and can subsequently provide current to deliver the secondary pulse during or immediately following the relative refractory period to provide PESP. For coupled pacing, following delivery of a PESP pulse during a first cardiac cycle, the passive recharge resistor is switched out of the circuit during the interval between cardiac cycles so that the capacitor can subsequently provide current to deliver the PESP pulse of the next cardiac cycle. In this regard, any of a variety of suitable high quality capacitors can be used that are capable of holding their charge state for several seconds, which is typically sufficient to allow an anodic pulse to be delivered during one cardiac cycle and then a cathodic pulse to be delivered during the next.

In some examples, an initial procedure is performed to set the pulse amplitudes and widths of the primary and secondary pulses using strength duration curves. For example, strength duration curves may be determined using the Lapicque equation for both a primary anodic pulse and a secondary cathodic pulse that relate pulse amplitudes as a function of pulse width and combined pulse voltages. A typical combined voltage is 4.0 V. To set the pulse widths, an iterative procedure is employed wherein, for a given width of the primary pulse, the width of the secondary pulse is incrementally increased while the combined voltage is held constant. The corresponding pulse amplitudes are derived from the strength duration curves (as represented, e.g. using a lookup table or functional equivalent) for both the primary and secondary pulses. A determination is then made as to whether both the primary and secondary absolute pulse amplitudes exceed a minimum target voltage by a predetermined safety margin. The minimum target voltage might be 1.0V with a safety margin of 1.0V (i.e., a 2:1 safety margin is required) so as to provide stimulation pulses of 2.0 V each.

If the absolute pulse amplitudes do not both exceed the target voltage by the predetermined safety margin, the pulse width of the primary anodic pulse is incrementally increased and the iterative procedure repeated. Often, the procedure will work to find some combination of pulse widths and amplitudes for the primary and secondary pulses sufficient to meet the given safety margin. If however, the pulse width of the primary pulse has been increased as far as permissible (as determined, for example, by programming limitations of the pacing device) without finding an acceptable combination of anodic and cathodic pulse parameters, then the combined voltage can be increased and the procedure repeated yet again at the higher voltage. Once the absolute amplitudes of the primary and secondary pulses both exceed the target voltage by the predetermined safety margin, paired or coupled pacing is then delivered by the implantable device using the lowest amplitudes and pulses widths that achieved the predetermined safety margins at the lowest combined voltage. In this manner, short pulse widths are found so as to reduce or minimize the amount of time during which a corresponding sensing channel is blanked.

Although described with respect to examples wherein the primary (or first) pulse is anodic and the secondary (or second) pulse is cathodic, alternative implementation might be exploited wherein the primary pulse is cathodic and the secondary pulse is anodic. Also, although the above summarized techniques track the refractory period, it should be understood that, in at least some embodiments, such tracking is not necessary. Instead, a system might measure changes in blood pressure to time the delivery of PESP pulses so as to optimize PESP without specifically measuring the refractory period.

### BRIEF DESCRIPTION OF THE DRAWINGS

Features and advantages of the described implementations can be more readily understood by reference to the following description taken in conjunction with the accompanying drawings.

FIG. 1 illustrates a pacing circuit for generating pacing pulses and PESP pluses as configured in accordance with the prior art;

FIG. 2 illustrates a biphasic (i.e. two-phase) stimulation pulse for use as a pacing pulse or a PESP pulse, which includes both cathodic and anodic phases in accordance with the prior art;

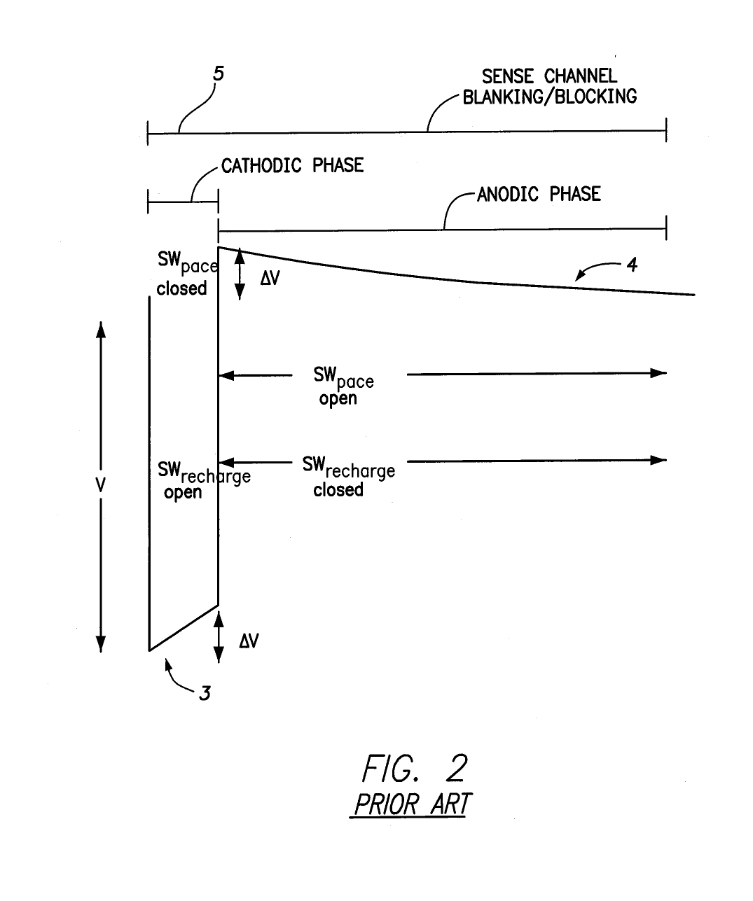

FIG. 3 illustrates a pair of biphasic stimulation pulses for use during paired pacing in accordance with the prior art;

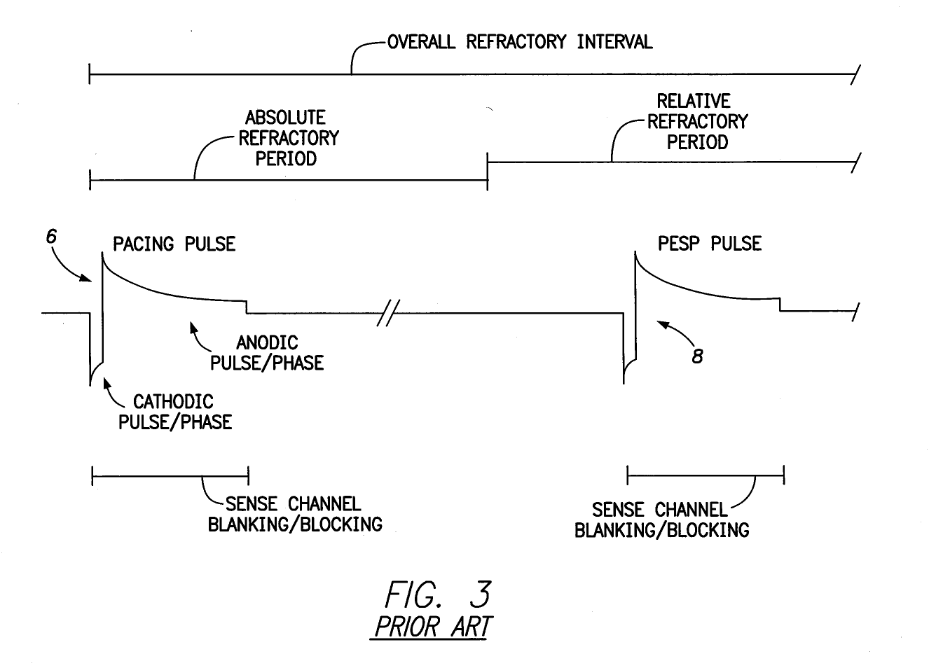

FIG. 4 illustrates components of an implantable medical system having a pacemaker, ICD or CRT device equipped to deliver split pulse PESP stimulation in accordance with an exemplary embodiment of the invention;

FIG. 5 summarizes a general technique for paired pacing that may be performed by the system of FIG. 4 wherein split pulse PESP stimulation is employed within a single cardiac cycle;

FIG. 6 illustrates a pair of single-phase stimulation pulses for use during paired pacing wherein the anodic and cathodic phases are separated by the absolute refractory period in accordance with the method of FIG. 5;

FIG. 7 summarizes a general technique for coupled pacing that may be performed by the system of FIG. 4 wherein split pulse PESP stimulation is delivered over consecutive cardiac cycles;

FIG. 8 illustrates a pair of single-phase stimulation pulses for use during coupled pacing wherein the anodic and cathodic phases are delivered during consecutive cardiac cycles in accordance with the method of FIG. 7;

FIG. 9 is a flowchart illustrating an exemplary implementation of the general method of FIG. 5 wherein both paired and coupled pacing are provided;

FIG. 10 illustrates a pacing circuit for generating pacing pulses and PESP pluses for use with the methods of FIGS. 5-9;

FIG. 11 is a flowchart illustrating an exemplary technique for use with the method of FIG. 9 wherein strength duration curves are employed to set the amplitudes and widths of the anodic and cathodic pulses;

FIG. 12 is a simplified, partly cutaway view, illustrating the device of FIG. 4 along with at set of leads implanted into the heart of the patient; and

FIG. 13 is a functional block diagram of the pacer/ICD of FIG. 12, illustrating basic circuit elements that provide cardioversion, defibrillation and/or pacing stimulation in the heart and particularly illustrating components for controlling the PESP stimulation techniques of FIGS. 5-11.

### DETAILED DESCRIPTION OF THE PREFERRED EMBODIMENTS

The following description includes the best mode presently contemplated for practicing the invention. This description is not to be taken in a limiting sense but is made merely to describe general principles of the invention. The scope of the invention should be ascertained with reference to the issued claims. In the description of the invention that follows, like numerals or reference designators will be used to refer to like parts or elements throughout.

### Overview of Implantable Systems and Methods

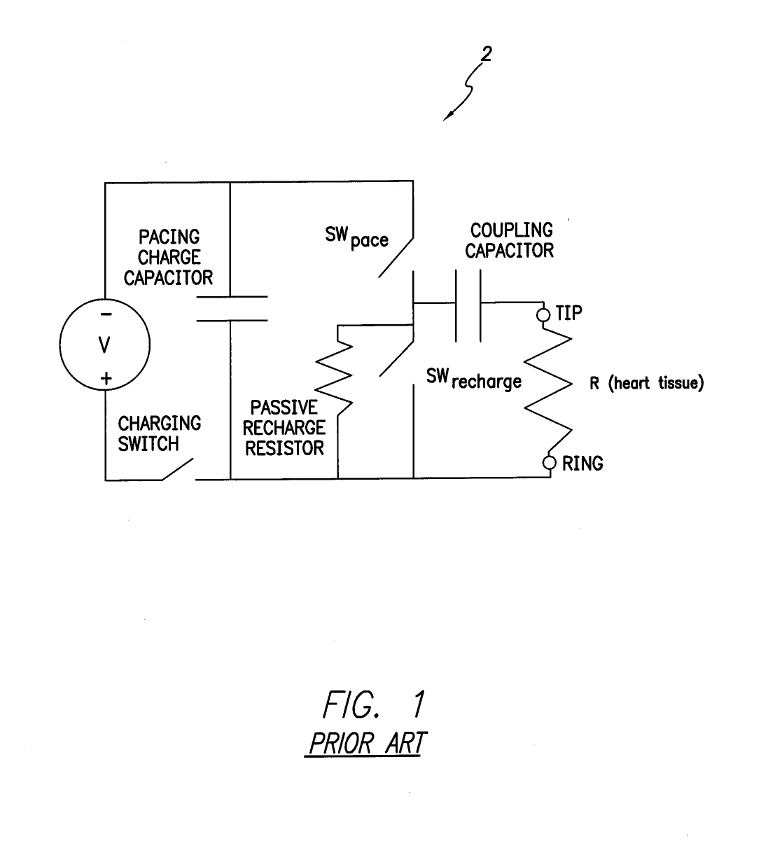

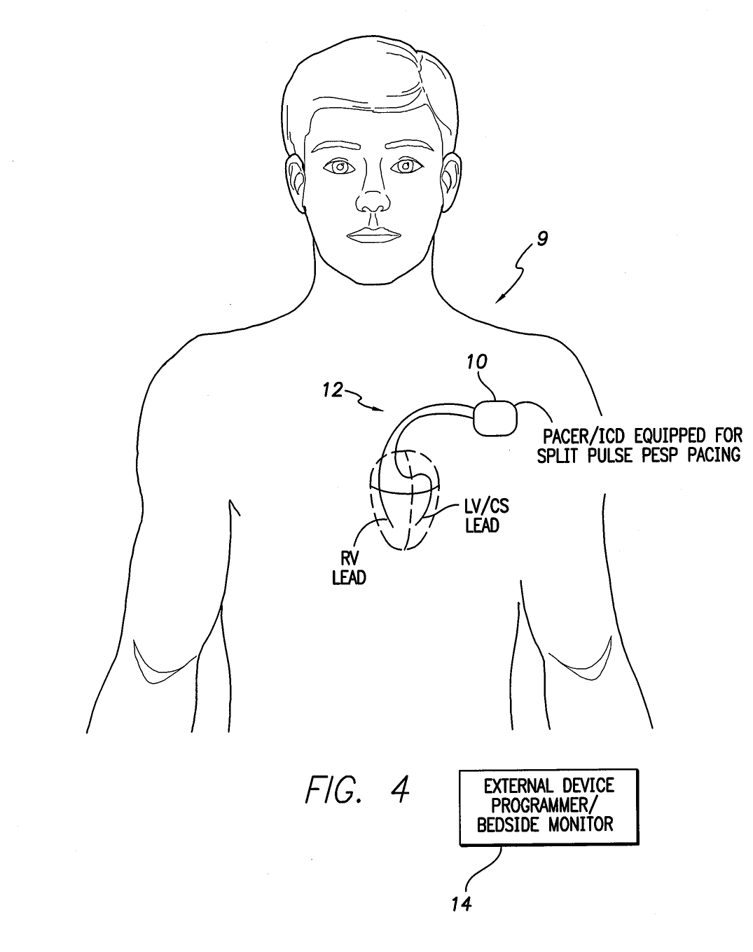

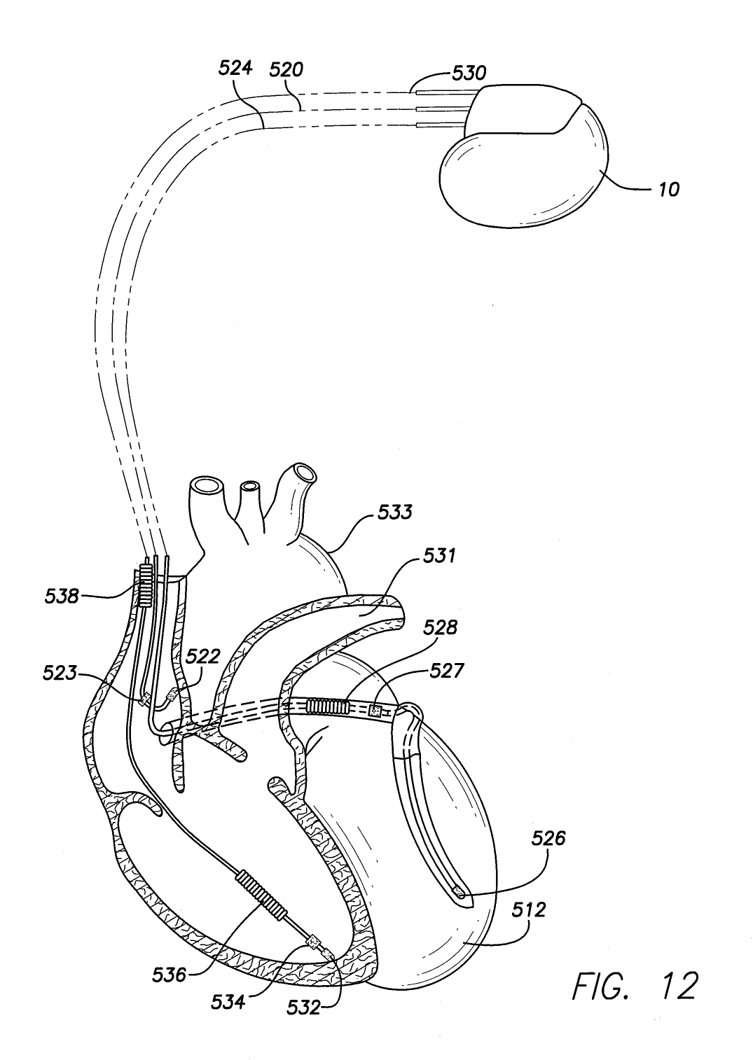

FIG. 4 illustrates an implantable medical system 9 capable of delivering PESP via paired or coupled pacing while using split or bifurcated anodic/cathodic stimulation pulses. In this example, the implantable medical system includes a pacer/ICD 10 or other cardiac stimulation device (such as a CRT device) equipped with a set of cardiac sensing/pacing leads 12 implanted on or within the heart of the patient, including at least an RV lead and an LV lead implanted via the coronary sinus (CS) for biventricular pacing. In FIG. 1, a stylized representation of the leads is set forth. A more accurate and complete illustration of the leads is provided within FIG. 12, discussed below. In the exemplary embodiments described herein, the PESP pacing is delivered using the LV and RV leads in accordance with biventricular pacing techniques.

The pacer/ICD is programmed using an external programming device 14 under clinician control. Programming commands can specify, for example, the amplitude and width of the anodic and cathodic pulses for use during PESP. At other times, the pacer/ICD may be in communication with a beside monitor or other diagnostic device such as a personal advisory module (PAM) that receives and displays data from the pacer/ICD, such as diagnostic data representative of the efficacy of PESP. In some embodiments, the bedside monitor is directly networked with a centralized computing system, such as the HouseCall™ system or the Merlin@home/Merlin.Net systems of St. Jude Medical, which can relay diagnostic information to the clinician.

Paired Pacing PESP with Split Anodic/Cathodic Pulses

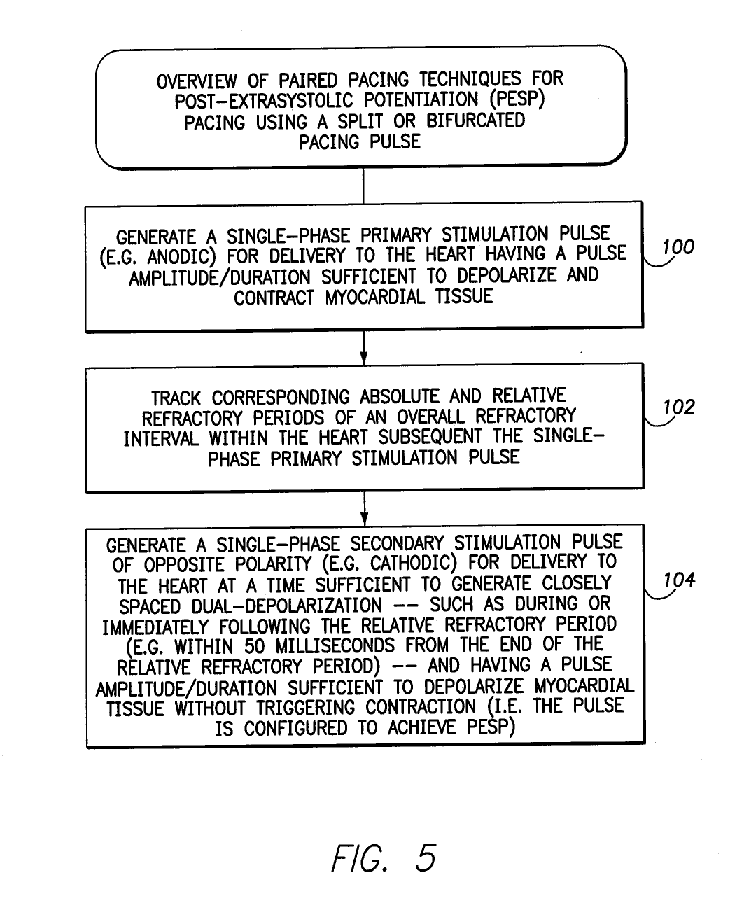

FIG. 5 illustrates techniques employed by the pacer/ICD of FIG. 4 (or other suitably-equipped systems) for controlling paired pacing using a split stimulation pulse. Beginning at step 100, the pacer/ICD generates a single-phase primary stimulation pulse (preferably anodic) for delivery to the heart of the patient using the leads, wherein the primary pulse has a pulse amplitude/duration sufficient to depolarize and contract myocardial tissue. At step 102, the pacer/ICD tracks the corresponding absolute and relative refractory periods of an overall refractory interval within the heart of the patient subsequent the single-phase primary stimulation pulse. Otherwise conventional techniques may be employed for tracking the refractory periods. Also, as already noted, in at least some embodiments, tracking of the refractory period is not necessary. Instead, other techniques may be used to time delivery of PESP such as measuring changes in blood pressure so as to optimize PESP without specifically measuring the refractory period. At step 104, the pacer/ICD generates a single-phase secondary stimulation pulse of opposite polarity (e.g. cathodic rather than anodic) for delivery to the heart at a time sufficient to generate closely spaced dual-depolarization such as during or immediately following the relative refractory period. The secondary stimulation pulse has a pulse amplitude/duration sufficient to depolarize myocardial tissue without triggering contraction (i.e. the pulse is configured to achieve PESP.)

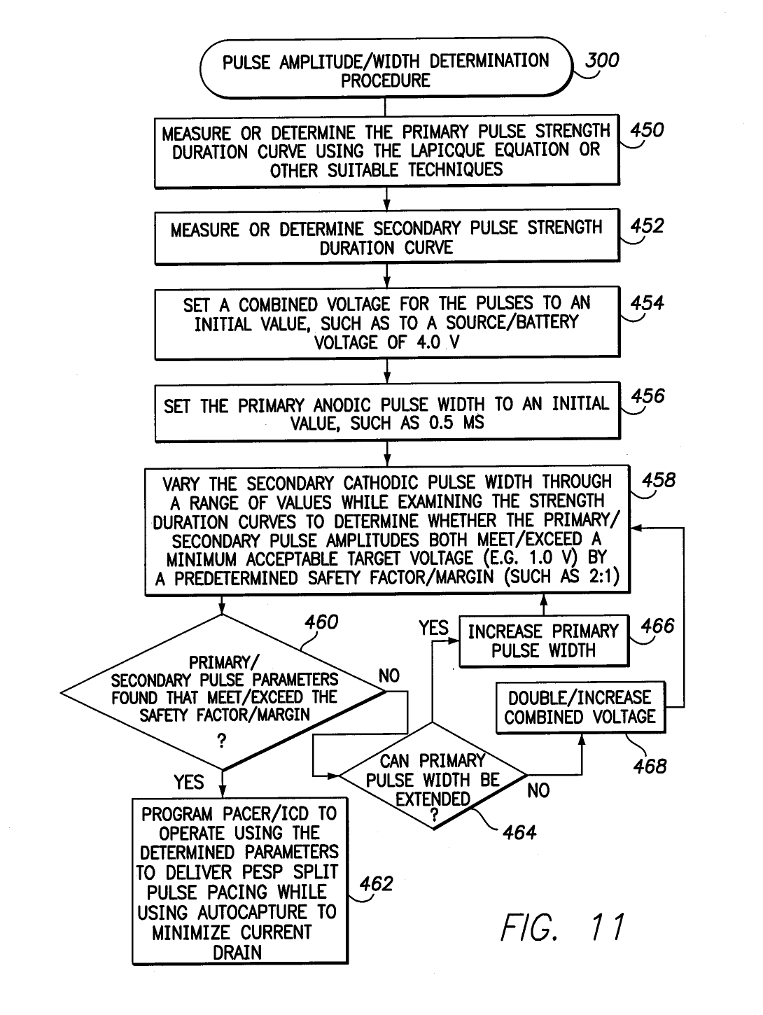

As already explained, during the absolute refractory period of an overall refractory interval, a second myocardial depolarization cannot be triggered because the myocardial tissue is not susceptible to further electrical stimulus. During or immediately beyond the relative refractory period, a second depolarization can be triggered with a sufficiently large stimulation pulse but not with a pulse of otherwise normal pulse amplitude. (As already noted, there is no sharp delineation between the relative refractory period and the non-refractory period. The threshold asymptotically approaches a minimum at the late diastolic threshold. The thresholds increase slightly as the cycle length shortens and then, at the relative refractory period, the thresholds start to climb dramatically into an absolute refractory period. Accordingly, the secondary stimulation pulse can be delivered late in or just “outside” the relative refractory period so as to generate closely spaced dual-depolarization.) In any case, the PESP pulse delivered at step 104 (in accordance with paired pacing) is configured to have a pulse amplitude and width to trigger depolarization without contraction. Techniques for setting the pulse amplitude and width of the secondary PESP pulse (as well as the primary stimulation pulse) are discussed below with reference to FIG. 11.

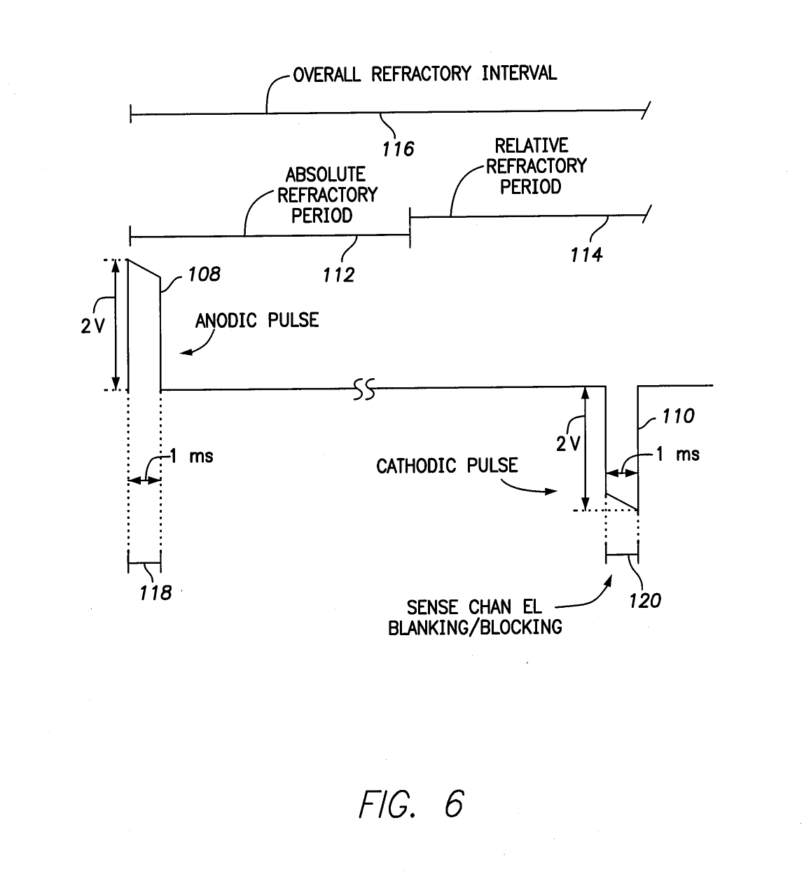

FIG. 6 illustrates an exemplary split or bifurcated stimulation pulse 106 for use with paired pacing having an anodic primary pulse 108 delivered to trigger depolarization and contraction followed by a cathodic secondary PESP pulse 110 delivered to trigger depolarization without contraction. The figure also illustrates the absolute refractory period 112 and the relative refractory period 114, which comprise the overall refractory interval 116. As can be seen, in this example the secondary pulse is delivered during the relative refractory period (though in other examples it might be delivered just beyond the end of the relative refractory period.) That is, the primary and secondary pulses are separated by at least the duration of the absolute refractory period. In this example, the primary and secondary pulses are each about 1 ms in duration and have a voltage of about 2 V. (Ranges of other suitable values are discussed below in connection with FIG. 11.)

Blanking/blocking intervals 118 and 120 for a corresponding sensing channel are also shown in FIG. 6. In this example, the blanking corresponds only to the period of time during delivery of the stimulation pulses, i.e. a total of 2 ms during the cardiac cycle. In other examples, blanking may extend somewhat beyond these intervals. In general, though, the amount of time during which blanking is performed using the techniques of FIGS. 5 and 6 is typically significantly less than that of conventional techniques that do not employ bifurcated pulses. As noted above, in a conventional PESP example, the primary stimulation pulse/phase has a duration within the range of 0.1 to 2 ms while the secondary pulse/phase has a duration within the range of 4 to 25 ms, yielding a total pulse duration of at least 6 ms and up to about to 27 ms, during which blanking is needed.

Note that, in the specific example of FIG. 6, the absolute refractory period is shown to begin immediately upon delivery of the primary pulse. Depending upon device programming, the absolute refractory period might instead be defined as beginning at some point later within the cardiac cycle, such as after completion of the primary pulse. In any case, otherwise conventional techniques can be employed for determining the time of the ending of the relative refractory so the PESP pulse may be delivered during or immediately beyond the relative refractory period. Note also that the timing of the secondary pulse within or immediately outside the relative refractory period (i.e. its timing relative to the beginning and end of the relative refractory period) can be set or determined in accordance with otherwise conventional PESP techniques while taking into account various factors.

The following patent documents discuss PESP therapy and related techniques: U.S. Pat. No. 7,184,833; U.S. Pat. No. 5,213,098; U.S. Pat. No. 7,289,850; U.S. Pat. No. 5,213,098; U.S. Patent Application 2007/0250122; U.S. Patent Application 2006/0149184; U.S. Patent Application 2006/0247698 and U.S. Patent Application 2007/0250122. See, also, Brunckhorst et al., “Cardiac Contractility Modulation by Non-Excitatory Currents: Studies In Isolated Cardiac Muscle”, Eur J Heart Fail. 2006 January; 8(1):7-15.

Coupled Pacing PESP with Split Anodic/Cathodic Pulses

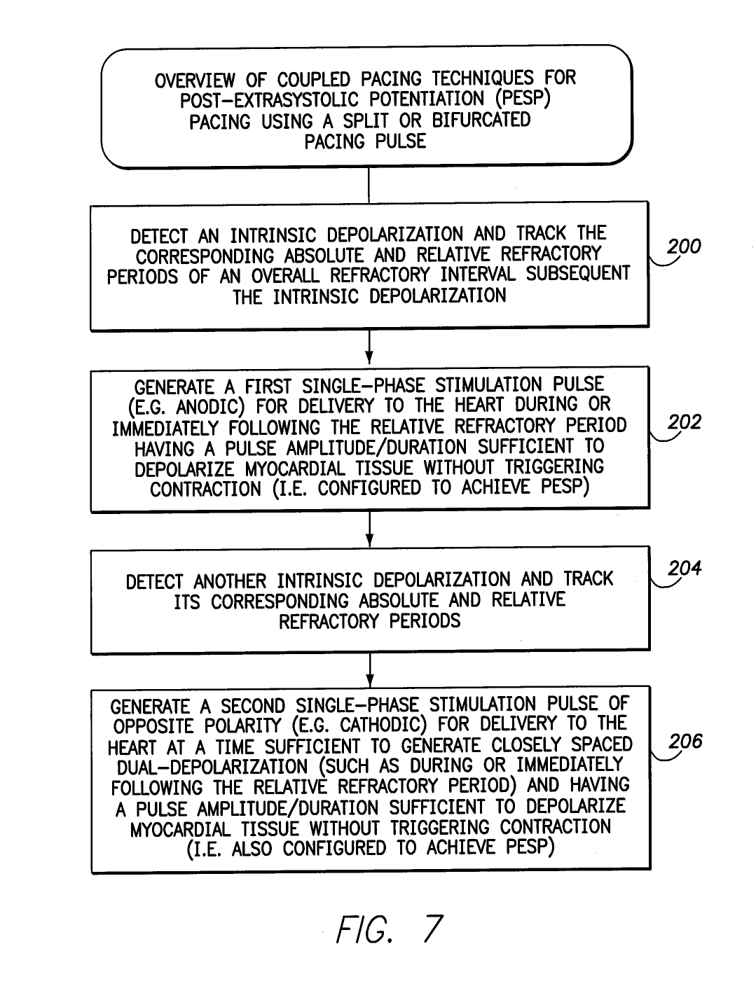

FIG. 7 illustrates techniques employed by the pacer/ICD of FIG. 4 (or other suitably-equipped systems) for controlling coupled pacing using a split or bifurcated stimulation pulse. Beginning at step 200, the pacer/ICD detects an intrinsic depolarization (i.e. a QRS complex or R-wave) and tracks the corresponding absolute and relative refractory periods of the overall refractory interval subsequent the intrinsic depolarization. Otherwise conventional techniques may be employed for detecting the intrinsic depolarization and tracking the refractory periods. At step 202, the pacer/ICD generates a first single-phase stimulation pulse (preferably anodic) for delivery to the heart during or immediately beyond the relative refractory period having a pulse amplitude/duration sufficient to depolarize myocardial tissue without triggering contraction (i.e. the pulse is configured to achieve PESP in accordance with coupled pacing.) At step 204, the pacer/ICD detects another intrinsic depolarization and tracks the subsequent corresponding absolute and relative refractory periods. At step 206, the pacer/ICD generates a second single-phase stimulation pulse of opposite polarity (e.g. cathodic) for delivery to the heart during or immediately following the relative refractory period. The second pulse likewise has a pulse amplitude/duration sufficient to depolarize myocardial tissue without triggering contraction (i.e. it is also configured to achieve PESP in accordance with coupled pacing.) As already noted, using this split process to deliver PESP has the primary benefit of minimizing the time that the sensing is blanked and blocked during the pacing pulses. Since the pulses can be in the range of 0.5 ms to 1.0 ms in duration, blanking and blocking may be limited to a few milliseconds for each pulse. This provides a greater alert period for detecting spontaneous events like PVCs.

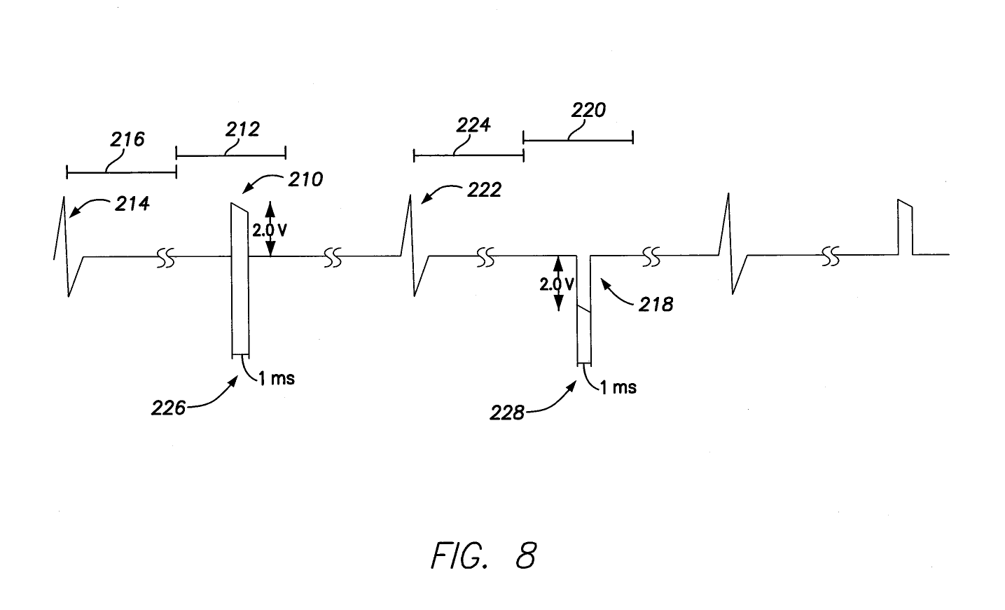

FIG. 8 illustrates exemplary split or bifurcated stimulation pulses for use with coupled pacing. During a first cardiac cycle, an anodic PESP pulse 210 is delivered to trigger depolarization without contraction during or immediately beyond the relative refractory 212 following a first intrinsic depolarization 214 and absolute refractory period 216. During a second cardiac cycle, another anodic PESP pulse 218 is delivered to trigger depolarization without contraction during or immediately beyond the relative refractory period 220 following a second intrinsic depolarization 222 and absolute refractory period 224. That is, the first and second PESP pulses 210 and 218 are within consecutive cardiac cycles. In this example, the first and second PESP pulses are each about 1 ms in duration and have absolute voltage magnitudes of about 2 V. (Again, ranges of other suitable values are discussed below in connection with FIG. 11.) Blanking/blocking intervals 226 and 228 for a corresponding sensing channel are also shown. In this example, as in the preceding example, the blanking corresponds only to the period of time during delivery of the stimulation pulses, i.e. a total of 2 ms during each pair of consecutive cardiac cycles. In other examples, blanking may extend somewhat beyond these intervals. In general, though, the amount of time during which blanking is performed using the technique of FIGS. 7 and 8 is typically significantly less than that of conventional techniques that do not employ bifurcated pulses.

PESP Example with Both Paired and Coupled Pacing

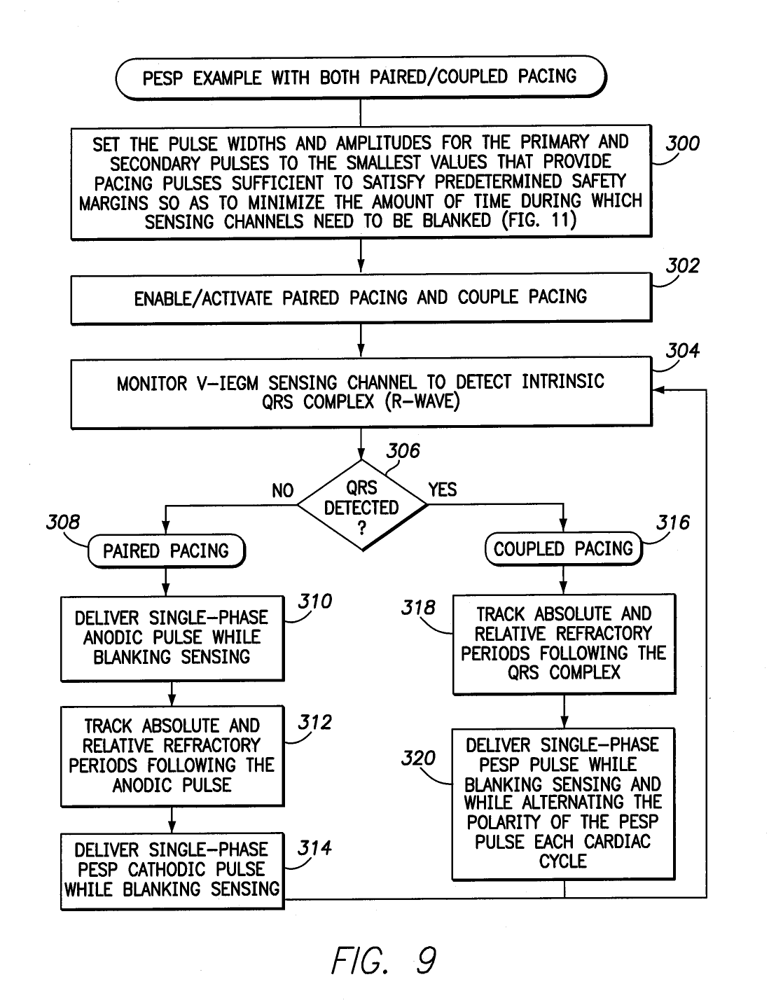

FIG. 9 illustrates an example wherein a pacer/ICD (or other suitably-equipped device) is equipped to provide both paired and coupled biventricular PESP pacing using split stimulation pulses. At step 300, the pacer/ICD sets the pulse widths and amplitudes for the primary and secondary pulses to the smallest values that provide pacing pulses sufficient to satisfy predetermined safety margins so as to minimize the amount of time during which sensing channels are blanked. Techniques for setting pulse amplitude and width to preferred or optimal values are discussed below with reference to FIG. 11. In some embodiments, the procedure for setting the pulse amplitude parameters is performed by an external system and the preferred parameters are programmed into the implanted device. In such embodiments, at step 300, the pacer/ICD merely retrieves the programmed values from device memory. In other embodiments, the pacer/ICD itself is equipped to perform the analysis using on-board components.

At step 302, the pacer/ICD enables or activates paired and coupled pacing (in accordance with commands previously entered by the clinician) and, at step 304, monitors the ventricular intracardiac electrogram (V-IEGM) sensing channel to detect intrinsic QRS complex (R-wave.) Assuming a QRS is not detected at step 306 within the current cardiac cycle, then paired PESP pacing is initiated, step 308. At step 310, the pacer/ICD delivers a single-phase anodic pulse while blanking sensing. At step 312, the pacer/ICD tracks absolute and relative refractory periods following the anodic pulse. At step 314, the pacer/ICD delivers a single-phase PESP cathodic pulse while blanking sensing. That is, during steps 308-314, the pacer/ICD performs the paired pacing PESP techniques of FIG. 5, as already described. Conversely, if a QRS is detected at step 306 within the current cardiac cycle, then coupled PESP pacing is initiated, step 316. At step 318, the pacer/ICD tracks absolute and relative refractory periods following the QRS complex. At step 320, the pacer/ICD delivers a single-phase PESP pulse while blanking sensing and while alternating the polarity of the PESP pulse each cardiac cycle. That is, during steps 316-320, the pacer/ICD performs the coupled pacing PESP techniques of FIG. 7, as already described.

It is noted that circumstances might arise within the patient where the pace/ICD switches from coupled pacing to paired pacing from one cardiac cycle to the next. As such, circumstances can arise where the pacer/ICD has just delivered an anodic PESP pulse during coupled pacing within one cardiac cycle but the next cardiac cycle requires paired pacing (which, as already explained, typically employs an anodic pulse as the primary pacing pulse.) Depending upon device programming, the pacer/ICD can either deliver a cathodic pulse as the primary pulse of paired pulse pair (followed by an anodic secondary pulse) or the device can reset its pacing circuit so as to allow for delivery of a second consecutive anodic pulse without an intervening cathodic pulse (by, for example, discharging the charge held on the coupling capacitor via a passive recharge/discharge resistor to thereby reset the circuit.)

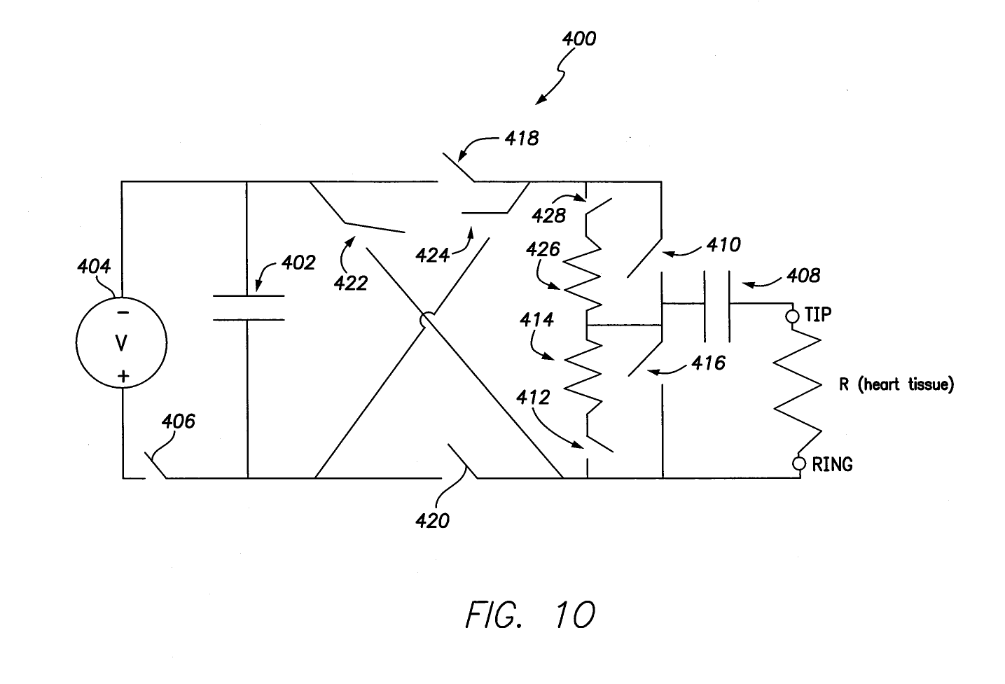

FIG. 10 illustrates a modified circuit 400 for generating split anodic/cathodic stimulation pulses. The operation of the circuit will be summarized in connection with paired pacing but it should be understood that a similar procedure can be employed during the delivery of coupled pacing. Charge for delivering the stimulation pulse is held in a pacing charge capacitor 402 based on voltage generated by a power source (e.g. battery 404) as controlled by a charging switch 406. A separate charge coupling capacitor 408 blocks direct current to the tip/ring electrodes during pacing to avoid electrode corrosion and to hold charge for delivering the second phase of the split anodic/cathodic pacing pulse. In order to deliver an anodic pulse as the primary pulse, switches 422 and 424 are closed while switches 418 and 420 are open. Assuming the pacing charge capacitor has been properly charged from voltage source 404, the delivery of the anodic phase of the stimulation pulse consists of closing switch 410 (SWpace) to provide a path for charge to flow from capacitor 402 into coupling capacitor 408 through the tip and ring pacing electrodes via heart tissue (which is represented by resistance R.) During this anodic process, which may last only 1 ms, the coupling capacitor (typically 5 microfarads) 408 accumulates a small amount of charge, Q=CΔV, subject to a small voltage, ΔV, which is only a fraction of the voltage of supply V. The anodic phase terminates by opening switch 410 (SWpace). If it is desired to make the primary anodal pulse larger and make the PESP cathodal pulse smaller, the passive recharge control switch 412 is closed while the control switch 428 remains open during at least a portion of the absolute refractory period. Increasing the duration that control switch 412 is closed while 428 remains open, increases the anodic primary pulse amplitude while decreasing the cathodic pulse amplitude. If it is desired to make the primary anodal pulse smaller and the cathodal pulse larger, the passive recharge switch 428 is closed at least a portion of the absolute and/or relative refractory period while the passive recharge switch 412 is remains open. Thus the relative amplitude of the anodal and cathodal pulse can be adjusted. The passive recharge resistors 426 and 414 can have a relatively high resistance of about 40 kilo-ohms.

Hence, the charge that accumulated on the coupling capacitor during the anodic phase remains on the capacitor during the absolute refractory period. The charge is then taken off the coupling capacitor during the cathodic phase delivered within or immediately beyond the relative refractory by closing recharge switch 416 (SWrecharge.) This phase may likewise last only 1 ms. If charge needs to be taken off the coupling capacitor, switch 412 while switch 428 remains open can be closed to allow for passive recharge via passive recharge resistor 414 after the PESP pulse is delivered. If charge needs to be put on the coupling capacitor, switch 428 can be closed while switch 412 remains open to allow for passive charging via passive recharge resistor 426 after the PESP pulse is delivered. The passive charge and recharge resistors 426 and 414 can have a relatively high resistance of about 40 kilo-ohms to allow for charging or for dissipation of residual charge during the period of time prior to the delivery of the next anodic primary pulse phase during the next paired pacing cardiac cycle. Thus the amplitude of the anodic pulse may be adjusted. Also, prior to the next cardiac cycle, the charging switch 406 is controlled to recharge the pacing charge capacitor 402 from the voltage source. Thereafter, during the next cardiac cycle, the overall process is repeated to deliver another split anodic/cathodic pulse pair. Note that the switches of the circuit are controlled by a microcontroller or other suitable control system to adjust timing of pulses and the amplitude of the pulses including a means of sensing the voltage on capacitor 408 so that charge and thus the voltage on capacitor may be adjusted (not shown in FIG. 10.) Note also that this is a simplified pacing circuit that only illustrates circuit components pertinent to this discussion. State-of-the-art pacing circuits can include numerous additional components. If it is desirable to deliver a cathodal pulse as the primary pulse and an anodal pulse for PESP, switches 420 and 418 are closed while switches 422 and 424 remain open. This inverts the voltage delivered from the pacing charge capacitor 402 and thus allows for inversion of the polarity primary and PESP pulses.

The operation of the circuit is similar during coupled pacing where, as already explained, the anodic pulse/phase is delivered during one cardiac cycle and the cathodic pulse/phase is delivered during the next. For coupled pacing, the passive recharge and charge resistors 414 and 426 are switched with 412 and 428 switches to adjust the amplitude of the anodic pulse during one cardiac cycle to adjust the amplitude of the cathodic pulse delivered during the next consecutive cardiac cycle (unless the circuit is reset in the interim to accommodate a switch from coupled pacing to paired pacing, as already noted.)

Hence, the pacing circuit of FIG. 1, discussed above, is modified as shown FIG. 10 to operate differently for the purposes of delivering split phase anodic/cathodic pulses. As discussed above the relative amplitudes of the anodal and cathodal pulses may be modified by adjusting the closing and opening of the passive recharge and charge resistors 414 and 426 using switches with 412 and 428. Alternatively the pulse amplitudes may be adjusted by modifying the first pacing phase duration relative the second phase duration that discharges the capacitor. For example, if the pacing phase and the recharge phase are equal in duration while switches 428 and 412 remain open, e.g. 0.5 ms for each phase, then the amplitude of the first phase and the amplitude of the second phase are substantially identical and sum to the source voltage V. Furthermore, as already noted, the pulses may be separated by relatively long durations since high quality capacitors will hold a charge state for at least several seconds. (This is true as long as the passive recharge resistor is switched out of the circuit, as described.) When switched in, the passive recharge resistors, typically 40 k, acts to provide a slow recharge or discharge with a time constant on the order 40K*5 microfarads=200 ms.

### Strength Duration Curve-Based Technique for Setting Pulse Amplitude/Width

FIG. 11 illustrates an exemplary technique for use at step 300 of FIG. 9 to set the pulse amplitudes and widths of the anodic and cathodic pulse phases of the split pulses. Briefly, this technique involves measuring the primary and secondary pulse thresholds at (at least) two different pulse widths and using the Lapicque equation to choose a pulse width and an amplitude for both the primary and secondary pulse that exceeds the amplitude of the strength duration curve by an appropriate safety factor. This may be achieved by choosing a voltage (for instance the battery voltage) then progressively increasing the primary pulse width while varying the secondary pulse until both pulses exceed the safety margin. If this criteria cannot be met at a given primary pulse width, the primary pulse width is increased and the secondary pulse width is varied until the criteria is met. The procedure may be iterated until both criteria are met. Finally, if neither pulse meets the criteria, the voltage is increased. Thus, this is a triple iterative process. Typically, 2:1 is the safety factor/ratio required for both the primary and secondary pulses to exceed the safety factors. As already noted, the procedure can be performed by the implanted device itself, if so equipped, or it can be performed in advance using an external system. In the following example, it is assumed that an external system performs the procedure.

Now, describing the technique in greater detail, at steps 450 and 452, the system measures, determined or inputs strength duration curves for the primary (anodic) and secondary (cathodic) pulse. The strength duration curves may be determined using the Lapicque Equation (or other suitable techniques) and represented using lookup tables or other functional equivalents.

Strength duration curves are discussed in, e.g.: U.S. Pat. No. 5,697,956 to Bornzin entitled “Implantable Stimulation Device having means for Optimizing Current Drain”; and in U.S. Pat. No. 7,574,259 to Pei, et al., entitled “Capture threshold and Lead Condition Analysis”; and U.S. Patent Application 2009/0270938 of Pei et al., also entitled “Capture Threshold and Lead Condition Analysis.” See, also, U.S. Pat. No. 6,738,668 to Mouchawar, et al., entitled “Implantable Cardiac Stimulation Device having a Capture Assurance System which Minimizes Battery Current Drain and Method for Operating the Same”; U.S. Pat. No. 6,615,082 to Mandell entitled “Method and Device for Optimally Altering Stimulation Energy to Maintain Capture of Cardiac Tissue”' and U.S. Pat. No. 5,692,907 to Glassel, et al., entitled “Interactive Cardiac Rhythm Simulator.”

The Lapicque Equation is discussed in aforementioned patents to Mouchawar, et al. (U.S. Pat. No. 6,738,668) and Mandell (U.S. Pat. No. 6,615,082) See, also, U.S. Pat. No. 6,549,806 to Kroll entitled “Implantable Dual Site Cardiac Stimulation Device having Independent Automatic Capture Capability” and U.S. Pat. No. 6,456,879 to Weinberg, entitled “Method and Device for Optimally Altering Stimulation Energy to Maintain Capture of Cardiac Tissue.”

At step 454, an initial voltage is selected (such as 4.0 V) and, at step 456, an initial width is selected for the primary anodic pulse (such as 0.5 ms.) Preferably, the initial width for the primary pulse is set well below a maximum programmable pulse width, where the maximum pulse width is specified, e.g., based on any programming restrictions of the pacer/ICD or other limiting factors. For example, if the primary pulse width can be programmed within the pacer/ICD through a range of values from 0.1 ms to 2.8 ms, then the initial width might be set to 0.5 ms.

At step 458, the system iteratively varies the secondary pulse width to determine voltages for the primary and secondary pulses from the strength duration relationship and then computes safety factors for both the primary and secondary pulses. That is, the device determines an amount by which the absolute magnitudes of the primary and secondary pulse amplitudes obtained from the strength duration relationship exceeds a minimum acceptable target voltage, such as 1.0 V, and compares this to a minimum acceptable safety margin, such as 1.0 V, to verify that the safety margin is met. For example, if the target voltage is 1.0 V and the safety factor is 1.0 V, the absolute magnitude of the pulse amplitudes for the primary and secondary pulses determined from the strength duration curve should both be at least 2.0 V. The safety factors can be expressed as a ratio of the resulting pulse amplitude to the minimum target voltage, such as a ratio of 2:1.

Table I provides exemplary strength duration relationship data:

TABLE I

| Anodic | Cathodic | Anodic | Cathodic |
|---|---|---|---|
| pulse | pulse | pulse | pulse |
| duration | duration | amplitude | amplitude |
| (ms) | (ms) | in volts | in volts |
| 0.5 | 0.1 | 0.7 | −3.3 |
| 0.5 | 0.2 | 1.1 | −2.9 |
| 0.5 | 0.3 | 1.5 | −2.5 |
| 0.5 | 0.4 | 1.8 | −2.2 |
| 0.5 | 0.5 | 2.0 | −2.0 |
| 0.5 | 0.6 | 2.2 | −1.8 |
| 0.5 | 0.7 | 2.3 | −1.7 |
| 0.5 | 0.8 | 2.5 | −1.5 |
| 0.5 | 0.9 | 2.6 | −1.4 |
| 0.5 | 1.0 | 2.7 | −1.3 |
| 0.5 | 1.2 | 2.8 | −1.2 |
| 0.5 | 1.4 | 2.9 | −1.1 |
| 0.5 | 1.6 | 3.0 | −1.0 |
| 0.5 | 1.8 | 3.1 | −0.9 |
| 0.5 | 2.0 | 3.2 | −0.8 |
| 0.5 | 2.4 | 3.3 | −0.7 |
| 0.5 | 2.8 | 3.3 | −0.7 |

The table provides an example where the source voltage is 4.0 V while the anodic pulse width is fixed at 0.5 ms and the cathodic pulse duration is varied from 0.1 to 2.8 ms. Note that the sum of the anodic and cathodic pulse voltages is 4.0 volts. This can be equal to the source (i.e. battery) voltage. In this particular example, the cathodic pulse amplitude can be represented by the equation:

Cathodic Pulse Amplitude=−0.1945*CD̂4+1.4208*CD̂3−3.8727*CD̂2+5.0746*CD−3.7487

where “CD” represents the cathodic pulse duration. Although Table I provides exemplary results when the anodic pulse is fixed at 0.5 ms, it should be understood that other combinations of values for other pulse widths can be predicted or determined mathematically for other anodic pulse widths.

Continuing with step 458 of FIG. 11, for the anodic pulse width of 0.5 ms, the system varies the cathodic pulse width from 0.1 ms to 2.8 ms while reading off the corresponding anodic and cathodic pulse amplitudes in an attempt to find a pair of values that both exceed the aforementioned safety factors. In this particular example, when the cathodic pulse width is 0.5 ms, the pulse amplitudes of the anodic and cathodic pulses both have absolute magnitudes of 2.0 V, which both exceed the target voltage of 1.0V by the safety factor ratio of 2:1. Accordingly, this particular combination of values is suitable for pacing in this particular example: anodic pulse width of 0.5 ms, cathodic pulse width of 0.5 ms, anodic pulse amplitude of 2.0 V, and cathodic pulse amplitude of −2.0 V.

Assuming that a suitable pair of primary and secondary pulse amplitudes/widths are found at step 458 that meet or exceed the safety factors (as verified at step 460), then the implantable device (e.g. pacer/ICD) is programmed at step 462 to operate at using the parameters. That is, the values are programmed into the device for use in delivering the aforementioned PESP split pulse pacing. Preferably, automatic capture techniques (i.e. AutoCapture™) are employed during pacing to minimize current drain. Automatic capture techniques are described, for example, in U.S. Pat. No. 6,731,985 to Poore, et al., entitled “Implantable Cardiac Stimulation System and Method for Automatic Capture Verification Calibration” and U.S. Pat. No. 5,697,956 to Bornzin, entitled “Implantable Stimulation Device having Means for Optimizing Current Drain.”

If, however, a combination of primary and secondary pulse amplitudes/widths cannot be found at step 458 where both the anodic and cathodic pulse amplitudes meet the safety factors despite varying the secondary pulse widths through a full range of programmable values, then at step 464 the system determines whether the primary width has been “maximized.” That is, the system determines whether the primary pulse width can still be increased from its currently selected value without exceeding its maximum permissible value. If it cannot be further increased, then the width has been maximized. Note that during a first iteration of the procedure, the primary pulse width will not yet be maximized since it is initially set to a value well below its maximum programmable value, as discussed above. Assuming, then, that the primary pulse width has not yet been maximized, the system incrementally increases the primary pulse width at step 466 and the iterative procedure of step 458 is repeated using the strength duration curve data corresponding to the new anodic pulse width. That is, a new table is generated or input that is similar to that of TABLE I but provides data for the new anodic pulse width and step 458 is repeated using the new table.

In the event that the primary pulse width is eventually maximized without finding a combination of pulse parameters that meet the safety factors, the combined voltage is increased at step 468 and the entire procedure repeated yet again. In the particular example of FIG. 11, the voltage is doubled at step 468, but other adjustment factors can be applied to the voltage.

Hence, FIG. 11 provides an exemplary technique for setting anodic and cathodic pulse parameters based on strength duration curve data. As explained, the relative amplitudes of the two pulses are mathematically predictable and a lookup table (or other suitable computational model) is used to predict the relative amplitudes and durations of the two pulses. If the system is instead designed to employ a cathodic pulse as the first phase, rather than an anodic pulse, similar techniques can be used to iterate anodic pulse while holding the cathodic pulse width fixed.

Thus, various techniques have been described for paired/coupled PESP pacing with split anodic/cathodic pulses. Although primarily described with respect to examples having a pacer/ICD, other implantable medical devices may be equipped to exploit the techniques described herein such as standalone CRT devices or CRT-D devices (i.e. a CRT device also equipped to deliver defibrillation shocks.) CRT and related therapies are discussed in, for example, U.S. Pat. No. 6,643,546 to Mathis et al., entitled “Multi-Electrode Apparatus and Method for Treatment of Congestive Heart Failure”; U.S. Pat. No. 6,628,988 to Kramer et al., entitled “Apparatus and Method for Reversal of Myocardial Remodeling with Electrical Stimulation”; and U.S. Pat. No. 6,512,952 to Stahmann et al., entitled “Method and Apparatus for Maintaining Synchronized Pacing”. See, also, U.S. Patent Application No. 2008/0306567 of Park et al., entitled “System and Method for Improving CRT Response and Identifying Potential Non-Responders to CRT Therapy” and U.S. Patent Application No. 2007/0179390 of Schecter, entitled “Global Cardiac Performance.”

Note that techniques described in U.S. patent application Ser. No. ______, filed ______, of Bornzin, entitled “Systems and Methods for Packed Pacing using Bifurcated Pacing Pulses of Opposing Polarity Generated by an Implantable Medical Device” (Atty. Docket A12P1046) may be exploited in at least some embodiments and this application is fully incorporated by reference herein (if filed prior hereto or contemporaneously herewith.)

For the sake of completeness, an exemplary pacer/ICD will now be described, which includes components for performing or controlling the functions and steps already described.

### Exemplary Pacer/ICD

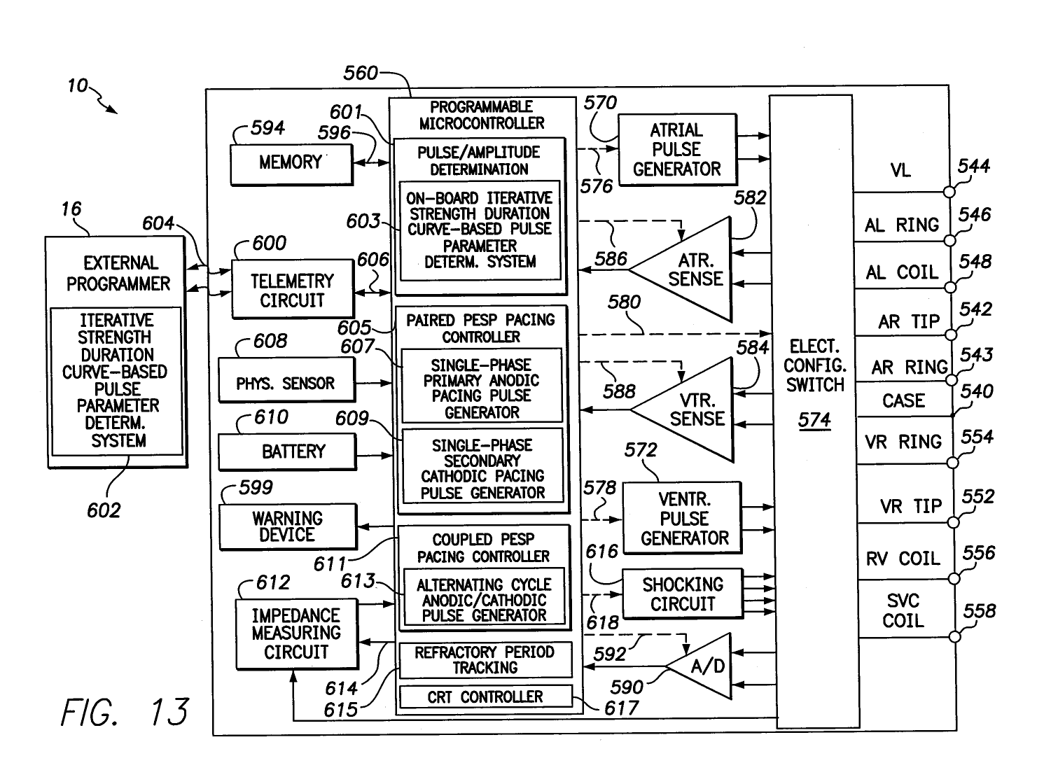

With reference to FIGS. 12 and 13, a description of an exemplary pacer/ICD will now be provided. FIG. 12 provides a simplified block diagram of the pacer/ICD, which is a dual-chamber stimulation device capable of treating both fast and slow arrhythmias with stimulation therapy, including cardioversion, defibrillation, and pacing stimulation, and also capable of providing split pulse PESP. To provide atrial chamber pacing stimulation and sensing, pacer/ICD 10 is shown in electrical communication with a heart 512 by way of a left atrial lead 520 having an atrial tip electrode 522 and an atrial ring electrode 523 implanted in the atrial appendage. Pacer/ICD 10 is also in electrical communication with the heart by way of a right ventricular lead 530 having, in this embodiment, a ventricular tip electrode 532, a right ventricular ring electrode 534, a right ventricular (RV) coil electrode 536, and a superior vena cava (SVC) coil electrode 538. Typically, the right ventricular lead 530 is transvenously inserted into the heart so as to place the RV coil electrode 536 in the right ventricular apex, and the SVC coil electrode 538 in the superior vena cava. Accordingly, the right ventricular lead is capable of receiving cardiac signals, and delivering stimulation in the form of pacing and shock therapy to the right ventricle.

To sense left atrial and ventricular cardiac signals and to provide left chamber pacing therapy, pacer/ICD 10 is coupled to a CS lead 524 designed for placement in the “CS region” via the CS os for positioning a distal electrode adjacent to the left ventricle and/or additional electrode(s) adjacent to the left atrium. As used herein, the phrase “CS region” refers to the venous vasculature of the left ventricle, including any portion of the CS, great cardiac vein, left marginal vein, left posterior ventricular vein, middle cardiac vein, and/or small cardiac vein or any other cardiac vein accessible by the CS. Accordingly, an exemplary CS lead 524 is designed to receive atrial and ventricular cardiac signals and to deliver left ventricular pacing therapy using at least a left ventricular tip electrode 526 and a LV ring electrode 525, left atrial pacing therapy using at least a left atrial ring electrode 527, and shocking therapy using at least a left atrial coil electrode 528. With this configuration, biventricular pacing can be performed. Although only three leads are shown in FIG. 12, it should also be understood that additional leads (with one or more pacing, sensing and/or shocking electrodes) might be used and/or additional electrodes might be provided on the leads already shown.

A simplified block diagram of internal components of pacer/ICD 10 is shown in FIG. 13. While a particular pacer/ICD is shown, this is for illustration purposes only, and one of skill in the art could readily duplicate, eliminate or disable the appropriate circuitry in any desired combination to provide a device capable of treating the appropriate chamber(s) with cardioversion, defibrillation and pacing stimulation. The housing 540 for pacer/ICD 10, shown schematically in FIG. 13, is often referred to as the “can”, “case” or “case electrode” and may be programmably selected to act as the return electrode for all “unipolar” modes. The housing 540 may further be used as a return electrode alone or in combination with one or more of the coil electrodes, 528, 536 and 538, for shocking purposes. The housing 540 further includes a connector (not shown) having a plurality of terminals, 542, 543, 544, 545, 546, 548, 552, 554, 556 and 558 (shown schematically and, for convenience, the names of the electrodes to which they are connected are shown next to the terminals). As such, to achieve right atrial sensing and pacing, the connector includes at least a right atrial tip terminal (A_{R} TIP) 542 adapted for connection to the atrial tip electrode 522 and a right atrial ring (A_{R} RING) electrode 543 adapted for connection to right atrial ring electrode 523. To achieve left chamber sensing, pacing and shocking, the connector includes at least a left ventricular tip terminal (V_{L} TIP) 544, a left ventricular ring terminal (V_{L} RING) 545, a left atrial ring terminal (A_{L} RING) 546, and a left atrial shocking terminal (A_{L} COIL) 548, which are adapted for connection to the left ventricular ring electrode 525, the left atrial ring electrode 527, and the left atrial coil electrode 528, respectively. To support right chamber sensing, pacing and shocking, the connector further includes a right ventricular tip terminal (V_{R} TIP) 552, a right ventricular ring terminal (V_{R} RING) 554, a right ventricular shocking terminal (V_{R} COIL) 556, and an SVC shocking terminal (SVC COIL) 558, which are adapted for connection to the right ventricular tip electrode 532, right ventricular ring electrode 534, the V_{R} coil electrode 536, and the SVC coil electrode 538, respectively.

At the core of pacer/ICD 10 is a programmable microcontroller 560, which controls the various modes of stimulation therapy. As is well known in the art, the microcontroller 560 (also referred to herein as a control unit) typically includes a microprocessor, or equivalent control circuitry, designed specifically for controlling the delivery of stimulation therapy and may further include RAM or ROM memory, logic and timing circuitry, state machine circuitry, and I/O circuitry. Typically, the microcontroller 560 includes the ability to process or monitor input signals (data) as controlled by a program code stored in a designated block of memory. The details of the design and operation of the microcontroller 560 are not critical to the invention. Rather, any suitable microcontroller 560 may be used that carries out the functions described herein. The use of microprocessor-based control circuits for performing timing and data analysis functions are well known in the art.

As shown in FIG. 13, an atrial pulse generator 570 and a ventricular pulse generator 572 generate pacing stimulation pulses for delivery by the right atrial lead 520, the right ventricular lead 530, and/or the CS lead 524 via an electrode configuration switch 574. It is understood that in order to provide stimulation therapy in each of the four chambers of the heart, the atrial and ventricular pulse generators, 570 and 572, may include dedicated, independent pulse generators, multiplexed pulse generators or shared pulse generators. The pulse generators, 570 and 572, are controlled by the microcontroller 560 via appropriate control signals, 576 and 578, respectively, to trigger or inhibit the stimulation pulses.

The microcontroller 560 further includes timing control circuitry (not separately shown) used to control the timing of such stimulation pulses (e.g., pacing rate, AV delay, atrial interconduction (inter-atrial) delay, or ventricular interconduction (V-V) delay, etc.) as well as to keep track of the timing of refractory periods, blanking intervals, noise detection windows, evoked response windows, alert intervals, marker channel timing, etc., which is well known in the art. Switch 574 includes a plurality of switches for connecting the desired electrodes to the appropriate I/O circuits, thereby providing complete electrode programmability. Accordingly, the switch 574, in response to a control signal 580 from the microcontroller 560, determines the polarity of the stimulation pulses (e.g., unipolar, bipolar, combipolar, etc.) by selectively closing the appropriate combination of switches (not shown) as is known in the art.

Atrial sensing circuits 582 and ventricular sensing circuits 584 may also be selectively coupled to the right atrial lead 520, CS lead 524, and the right ventricular lead 530, through the switch 574 for detecting the presence of cardiac activity in each of the four chambers of the heart. Accordingly, the atrial (ATR. SENSE) and ventricular (VTR. SENSE) sensing circuits, 582 and 584, may include dedicated sense amplifiers, multiplexed amplifiers or shared amplifiers. The switch 574 determines the “sensing polarity” of the cardiac signal by selectively closing the appropriate switches, as is also known in the art. In this way, the clinician may program the sensing polarity independent of the stimulation polarity. Each sensing circuit, 582 and 584, preferably employs one or more low power, precision amplifiers with programmable gain and/or automatic gain control, bandpass filtering, and a threshold detection circuit, as known in the art, to selectively sense the cardiac signal of interest. The automatic gain control enables pacer/ICD 10 to deal effectively with the difficult problem of sensing the low amplitude signal characteristics of atrial or ventricular fibrillation. The outputs of the atrial and ventricular sensing circuits, 582 and 584, are connected to the microcontroller 560 which, in turn, are able to trigger or inhibit the atrial and ventricular pulse generators, 570 and 572, respectively, in a demand fashion in response to the absence or presence of cardiac activity in the appropriate chambers of the heart.

For arrhythmia detection, pacer/ICD 10 utilizes the atrial and ventricular sensing circuits, 582 and 584, to sense cardiac signals to determine whether a rhythm is physiologic or pathologic. As used in this section, “sensing” is reserved for the noting of an electrical signal, and “detection” is the processing of these sensed signals and noting the presence of an arrhythmia. The timing intervals between sensed events (e.g., AS, VS, and depolarization signals associated with fibrillation which are sometimes referred to as “F-waves” or “Fib-waves”) are then classified by the microcontroller 560 by comparing them to a predefined rate zone limit (i.e., bradycardia, normal, atrial tachycardia, atrial fibrillation, low rate VT, high rate VT, and fibrillation rate zones) and various other characteristics (e.g., sudden onset, stability, physiologic sensors, and morphology, etc.) in order to determine the type of remedial therapy that is needed (e.g., bradycardia pacing, antitachycardia pacing, cardioversion shocks or defibrillation shocks).

Cardiac signals are also applied to the inputs of an analog-to-digital (A/D) data acquisition system 590. The data acquisition system 590 is configured to acquire intracardiac electrogram signals, convert the raw analog data into a digital signal, and store the digital signals for later processing and/or telemetric transmission to an external device 16. The data acquisition system 590 is coupled to the right atrial lead 520, the CS lead 524, and the right ventricular lead 530 through the switch 574 to sample cardiac signals across any pair of desired electrodes. The microcontroller 560 is further coupled to a memory 594 by a suitable data/address bus 596, wherein the programmable operating parameters used by the microcontroller 560 are stored and modified, as required, in order to customize the operation of pacer/ICD 10 to suit the needs of a particular patient. Such operating parameters define, for example, the amplitude or magnitude, pulse duration, electrode polarity, for both pacing pulses and impedance detection pulses as well as pacing rate, sensitivity, arrhythmia detection criteria, and the amplitude, waveshape and vector of each shocking pulse to be delivered to the patient's heart within each respective tier of therapy. Other pacing parameters include base rate, rest rate and circadian base rate.

Advantageously, the operating parameters of the implantable pacer/ICD 10 may be non-invasively programmed into the memory 594 through a telemetry circuit 600 in telemetric communication with the external device 16, such as a programmer, transtelephonic transceiver or a diagnostic system analyzer. The telemetry circuit 600 is activated by the microcontroller by a control signal 606. The telemetry circuit 600 advantageously allows intracardiac electrograms and status information relating to the operation of pacer/ICD 10 (as contained in the microcontroller 560 or memory 594) to be sent to the external device 16 through an established communication link 604. Pacer/ICD 10 further includes an accelerometer or other physiologic sensor or sensors 608, sometimes referred to as a “rate-responsive” sensor because it is typically used to adjust pacing stimulation rate according to the exercise state of the patient.

However, physiological sensor(s) 608 can be equipped to sense any of a variety of cardiomechanical parameters, such as heart sounds, systemic pressure, etc. As can be appreciated, at least some these sensors may be mounted outside of the housing of the device and, in many cases, will be mounted to the leads of the device. Moreover, the physiological sensor 608 may further be used to detect changes in cardiac output, changes in the physiological condition of the heart, or diurnal changes in activity (e.g., detecting sleep and wake states) and to detect arousal from sleep. Accordingly, the microcontroller 560 responds by adjusting the various pacing parameters (such as rate, AV delay, V-V delay, etc.) at which the atrial and ventricular pulse generators, 570 and 572, generate stimulation pulses. While shown as being included within pacer/ICD 10, it is to be understood that the physiologic sensor 608 may also be external to pacer/ICD 10, yet still be implanted within or carried by the patient. A common type of rate responsive sensor is an activity sensor incorporating an accelerometer or a piezoelectric crystal and/or a 3D-accelerometer capable of determining the posture within a given patient, which is mounted within the housing 540 of pacer/ICD 10. Other types of physiologic sensors are also known, for example, sensors that sense the oxygen content of blood, respiration rate and/or minute ventilation, pH of blood, ventricular gradient, etc.

The pacer/ICD additionally includes a battery 610, which provides operating power to all of the circuits shown in FIG. 13. The battery 610 may vary depending on the capabilities of pacer/ICD 10. If the system only provides low voltage therapy, a lithium iodine or lithium copper fluoride cell typically may be utilized. For pacer/ICD 10, which employs shocking therapy, the battery 610 should be capable of operating at low current drains for long periods, and then be capable of providing high-current pulses (for capacitor charging) when the patient requires a shock pulse. The battery 610 should also have a predictable discharge characteristic so that elective replacement time can be detected. Accordingly, appropriate batteries are employed.

As further shown in FIG. 13, pacer/ICD 10 is shown as having an impedance measuring circuit 612, which is enabled by the microcontroller 560 via a control signal 614. Uses for an impedance measuring circuit include, but are not limited to, lead impedance surveillance during the acute and chronic phases for proper lead positioning or dislodgement; detecting operable electrodes and automatically switching to an operable pair if dislodgement occurs; measuring respiration or minute ventilation; measuring thoracic impedance for determining shock thresholds; detecting when the device has been implanted; measuring respiration; and detecting the opening of heart valves, etc. The impedance measuring circuit 612 is advantageously coupled to the switch 674 so that any desired electrode may be used.

In the case where pacer/ICD 10 is intended to operate as an implantable cardioverter/defibrillator (ICD) device, it detects the occurrence of an arrhythmia, and automatically applies an appropriate electrical shock therapy to the heart aimed at terminating the detected arrhythmia. To this end, the microcontroller 560 further controls a shocking circuit 616 by way of a control signal 618. The shocking circuit 616 generates shocking pulses of low (up to 0.5 joules), moderate (0.5-10 joules) or high energy (11 to 40 joules or more), as controlled by the microcontroller 560. Such shocking pulses are applied to the heart of the patient through at least two shocking electrodes, and as shown in this embodiment, selected from the left atrial coil electrode 528, the RV coil electrode 536, and/or the SVC coil electrode 538. The housing 540 may act as an active electrode in combination with the RV electrode 536, or as part of a split electrical vector using the SVC coil electrode 538 or the left atrial coil electrode 528 (i.e., using the RV electrode as a common electrode). Cardioversion shocks are generally considered to be of low to moderate energy level (so as to minimize pain felt by the patient), and/or synchronized with an R-wave and/or pertaining to the treatment of tachycardia. Defibrillation shocks are generally of moderate to high energy level (i.e., corresponding to thresholds in the range of 6-40 joules), delivered asynchronously (since R-waves may be too disorganized), and pertaining exclusively to the treatment of fibrillation. Accordingly, the microcontroller 560 is capable of controlling the synchronous or asynchronous delivery of the shocking pulses.

Insofar as PESP pacing is concerned, the microcontroller includes a pulse/amplitude determination system 601 having, in this example, an on-board iterative strength duration curve-based pulse parameter determination system 603 operative to set the primary and secondary pulse amplitudes and widths using techniques discussed above. As noted, in some implementations, the determination is instead made by an external system with the pulse parameters then programmed into the pacer/ICD via telemetry. This alternative embodiment is illustrated by way of the iterative strength duration curve-based pulse parameter determination system 602 of external programmer 16. In circumstances where the external system determines the values and then programs the pacer/ICD, the pulse/amplitude determination system 601 of the pacer/ICD retrieves the programmed parameters from memory 594 prior to delivery of PESP pacing.

To control or provide for paired PESP pacing, the microcontroller includes a paired PESP pacing controller 605, which includes a single-phase primary anodic pacing pulse generator 607 for generating/controlling the primary pulses and a single-phase secondary cathodic pacing pulse generator 609 for generating/controlling the secondary pulses, using techniques described above. To control or provide for coupled PESP pacing, the microcontroller includes a coupled PESP pacing controller 611, which includes an alternating cycle anodic/cathodic pulse generator 613 for generating/controlling the delivery of alternating single-phase anodic and cathodic pulses during alternating cardiac cycles, as described above. Absolute and relative refractory periods are tracked using refractory period tracking system 615. CRT pacing can be controlled using a CRT controller 617. Any diagnostic data pertinent to PESP pacing can be stored in memory 594 for eventual transmission to an external system. In the event any warnings are needed, such as warning pertaining to PESP pacing, such warnings can be delivered using an onboard warning device, which may be, e.g., a vibrational device or a “tickle” voltage warning device.

Depending upon the implementation, the various components of the microcontroller may be implemented as separate software modules or the modules may be combined to permit a single module to perform multiple functions. In addition, although shown as being components of the microcontroller, some or all of these components may be implemented separately from the microcontroller, using application specific integrated circuits (ASICs) or the like.

In general, while the invention has been described with reference to particular embodiments, modifications can be made thereto without departing from the scope of the invention. Note also that the term “including” as used herein is intended to be inclusive, i.e. “including but not limited to.”

What is claimed is:

1. A method for use with an implantable cardiac stimulation device equipped to deliver postextrasystolic potentiation (PESP) pacing, the method comprising:
generating a single-phase primary stimulation pulse for delivery to the heart of the patient sufficient to depolarize myocardial tissue, the single-phase primary pulse being one of anodic or cathodic; and
generating a single-phase secondary stimulation pulse for delivery to the heart of the patient at a time sufficient to generate closely spaced dual-depolarization, the secondary pulse being opposite in polarity to the primary pulse and being configured to achieve PESP.

2. The method of claim 1 wherein the single-phase secondary stimulation pulse is delivered during a relative refractory period.

3. The method of claim 2 wherein the single-phase secondary stimulation pulse is delivered immediately following a relative refractory period.

4. The method of claim 3 wherein the single-phase secondary stimulation pulse is delivered within 50 milliseconds from the end of the relative refractory period.

5. The method of claim 1 wherein the single-phase primary pulse is an anodic pulse and the single-phase secondary pulse is a cathodic pulse.

6. The method of claim 5 wherein the single-phase primary pulse and the single-phase secondary pulse have about equal voltages and about equal durations.

7. The method of claim 6 wherein the single-phase primary pulse has an pulse amplitude of about 2 volts and a duration of about 1 millisecond (ms) and wherein the single-phase secondary pulse also has a pulse amplitude of about 2 volts and a duration of about 1 ms.

8. The method of claim 5 further including a preliminary step of setting pulse amplitudes and pulse widths for the single-phase primary pulse and for the single-phase secondary pulse.

9. The method of claim 8 wherein the preliminary step of setting the pulse amplitudes and pulse widths comprises:
determining strength duration curves for the primary anodic pulse and for the secondary cathodic pulse that relate pulse amplitudes as a function of pulse width and combined voltages;
setting a combined voltage to a starting voltage;
selecting a starting pulse width for the primary anodic pulse; and
iteratively setting the pulse width of the secondary cathodic pulse by incrementally increasing the width of the secondary cathodic pulse while holding the combined voltage substantially constant and while determining whether corresponding pulse amplitudes for the primary pulse and the secondary pulse obtained from the strength duration curves both exceed a minimum target voltage by a predetermined safety margin.

10. The method of claim 9 wherein, if the pulse amplitudes do not both exceed the target voltage by the predetermined safety margin, incrementally increasing the pulse width of the primary anodic pulse up to a maximum programmable width while repeating the step of iteratively setting the pulse width of the secondary cathodic pulse.

11. The method of claim 10 wherein, if the primary anodic pulse has reached its maximum programmable width without resulting amplitudes of the primary anodic pulse and the secondary cathodic pulse both exceeding the target voltage by the predetermined safety margin, increasing the combined voltage above the starting voltage and repeating the step of iteratively setting the pulse width of the secondary cathodic pulse.

12. The method of claim 9 wherein, once the amplitudes of the primary anodic pulse and the secondary cathodic pulse both exceed the target voltage by the predetermined safety margin, delivering pacing using the lowest amplitudes and pulses widths that achieved the predetermined safety margins at the lowest combined voltage.

13. The method of claim 9 wherein the safety margin is 2:1 as represented as a ratio of pulse amplitude to minimum target voltage.

14. The method of claim 9 wherein the strength duration curves are determined using the Lapicque equation.

15. The method of claim 9 wherein the strength duration curves are represented using one or more of a: lookup table or a functional equivalent to a lookup table.

16. The method of claim 5 wherein the refractory interval also includes an absolute refractory period prior to the relative refractory period.

17. The method of claim 5 for use with a device equipped to blank a sensing channel during delivery of stimulation pulses and wherein a width of the single-phase primary stimulation pulse and a width of the single-phase secondary PESP pulse are set to reduce an amount of time during which sensing channel blanking is employed.

18. The method of claim 1 wherein the steps of generating the single-phase primary stimulation pulse and generating the single-phase secondary stimulation pulse are performed in response to a paced depolarization in accordance with paired pacing.

19. The method of claim 1 for use with a device having a pacing circuit including a passive recharge resistor and having at least one capacitor and wherein, between generation of the primary pulse and the secondary pulse of opposite polarity, the passive recharge resistor is decoupled from the capacitor to prevent discharge of the capacitor.

20. The method of claim 1 wherein the single-phase primary pulse is a cathodic pulse and the single-phase secondary pulse is an anodic pulse.

21. A system for use with an implantable cardiac stimulation device equipped to deliver postextrasystolic potentiation (PESP) pacing, the system comprising:
a single-phase primary stimulation pulse generator operative to generate a single-phase primary stimulation pulse for delivery to the heart of the patient, the single-phase primary pulse being one of anodic or cathodic;
a single-phase secondary stimulation pulse generator operative to generate a single-phase secondary stimulation pulse for delivery to the heart of the patient at a time sufficient to generate closely spaced dual-depolarization, the single-phase secondary stimulation pulse being opposite in polarity to the primary pulse and configured to achieve PESP.

22. The system of claim 21 further including a strength duration curve-based system operative to set pulse amplitudes and pulse widths for the single-phase primary pulse and for the single-phase secondary pulse based on predetermined strength duration curves for the primary pulse and for the secondary pulse.

23. The system of claim 21 wherein the primary stimulation pulse generator, the refractory period tracking system, and the secondary stimulation pulse generator are components of a paired pacing system operative in response to detection of a paced depolarization.

24. The system of claim 21 wherein the refractory period tracking system and the secondary stimulation pulse generator are components of a coupled pacing system operative in response to detection an intrinsic depolarization.

25. A method for use with an implantable cardiac stimulation device equipped to deliver postextrasystolic potentiation (PESP) pacing, the method comprising:
detecting a first intrinsic depolarization and tracking a corresponding first refractory interval including a first relative refractory period;
generating a first single-phase stimulation pulse for delivery to the heart of the patient timed relative to the first relative refractory period to generate closely spaced dual-depolarization, the first single-phase stimulation pulse being configured to achieve PESP;
detecting a second intrinsic depolarization and tracking a corresponding second refractory interval including a second relative refractory period; and
generating a second single-phase stimulation pulse for delivery to the heart of the patient timed relative to the second relative refractory period to generate closely spaced dual-depolarization, the second stimulation pulse being opposite in polarity to a polarity of the first stimulation pulse and configured to achieve PESP.

26. The method of claim 21 wherein the first single-phase stimulation pulse is an anodic pulse and the second single-phase stimulation is a cathodic pulse.

27. The method of claim 22 wherein the first single-phase stimulation pulse is a cathodic pulse and the second single-phase stimulation is an anodic pulse.

28. A system for use with an implantable cardiac stimulation device equipped to deliver postextrasystolic potentiation (PESP) pacing, the system comprising:
a refractory interval tracking system operative to track refractory intervals within the heart of the patient subsequent to intrinsic depolarization events, the refractory intervals including relative refractory periods; and
an alternating cycle anodic/cathodic pulse generator operative to generate a first single-phase stimulation pulse for delivery to the heart of the patient timed relative to a first relative refractory period sufficient to generate closely spaced dual-depolarization following a first intrinsic depolarization event within a first cardiac cycle and further operative to generate a second single-phase stimulation pulse for delivery to the heart of the patient timed relative to a second relative refractory period following a second intrinsic depolarization event within a second cardiac cycle at a time sufficient to generate another closely spaced dual-depolarization, the first and single-phase stimulation pulses both being configured to achieve PESP.
